(19) United States
(12) Patent Application Publication
(10) Pub. No.: US 2004/0176290 A1
(43) Pub. Date: Sep. 9, 2004

(54) Anti-angiogenic state in mice and humans with retinal photorecptor cell degeneration

(76) Inventors: Renata Pasqualini, Houston, TX (US); Wadih Arap, Houston, TX (US)

Correspondence Address: David L Parker, Fulbright & Jaworski, Suite 2400, 600 Congress Avenue, Austin, TX, 78701, US

(21) Appl. No.: 10/484,550

(22) PCT Filed: Jul. 17, 2002
(86) PCT No.: PCT/US02/22971

Related U.S. Application Data
(60) Provisional application No. 60/306,506, filed on Jul. 18, 2001.

(51) Int. Cl. A61K 38/17; A61K 31/65; A61K 31/515; A61K 31/24

(52) U.S. Cl. 514/12; 606/2; 514/152; 514/270; 514/535

(57) ABSTRACT

Neonatal mice with classic inherited retinal degeneration (Pdeb^{rd1}/Pdeb^{rd1}) are disclosed which fail to mount reactive retinal neovascularization in a mouse model of oxygen-induced proliferative retinopathy. Also disclosed is a comparable human paradigm: spontaneous regression of retinal neovascularization associated with long-standing diabetes mellitus which occurs when retinitis pigmentosa becomes clinically evident. Both mouse and human data indicate that reactive retinal neovascularization either fails to develop or regresses when the number of photoreceptor cells is markedly reduced. The results show that a functional mechanism underlying this anti-angiogenic state is failure of the predicted up-regulation of vascular endothelial growth factor (VEGF), although other growth factors may also be involved. Preventive and therapeutic methods useful against both proliferative and degenerative retinopathies are also disclosed.

### BACKGROUND OF THE INVENTION

This application claims the benefit under 35 U.S.C. § 119(e) of U.S. Provisional Patent Application No. 60/306,506, filed Jul. 18, 2001. This invention was made with government support under grants DAMD 17-98-1-8041 and 17-98-1-8581 from the U.S. Army and grants 1R01CA78512-01A1, 1R1CA90810-01 and 1R01CA82976-01 from the National Institutes of Health. The government has certain rights in this invention.

### FIELD OF THE INVENTION

The invention relates to the field of animal models and methods of treating vascular diseases. More particularly, the invention relates to a mouse model with oxygen-induced ischemic retinopathy. Other aspects of the invention concern methods and compositions for treating and/or preventing retinal neovascularization, retinopathy of prematurity and diabetic retinopathy.

### BACKGROUND OF THE INVENTION

Abnormal angiogenesis accompanies many pathological conditions including cancer, inflammation and eye diseases. Proliferative retinopathy due to retinal neovascularization is a leading cause of blindness in developed countries. Another major cause of irreversible visual loss is retinitis pigmentosa, a group of diseases characterized by progressive photoreceptor cell degeneration. Anecdotal evidence suggests that proliferative diabetic retinopathy is rarely associated clinically with retinitis pigmentosa.

Excessive formation of new blood vessels in the retina is considered a hallmark of ischemic retinopathies such as diabetic retinopathy, a leading cause of blindness in the United States and Europe. Moreover, ocular neovascularization is considered a common etiological factor in diseases ranging widely in age of onset, from retinopathy of prematurity in oxygen-treated infants, to sickle cell disease and retinal venous occlusions seen in adults, to age-related macular degeneration observed in the elderly (Neeley et al, Am. J. Pathol., 53:665-670, 1998; Folkman et al., Cell, 87:1153-1155, 1996). Present methods for treating and/or preventing retinopathies are ineffective.

### SUMMARY OF THE INVENTION

Neonatal mice with classic inherited retinal degeneration (Pdeb^{rd1}/Pdeb^{rd1}) are disclosed which fail to mount reactive retinal neovascularization in a mouse model of oxygen-induced proliferative retinopathy. Also disclosed is a comparable human paradigm: spontaneous regression of retinal neovascularization associated with long-standing diabetes mellitus that occurs when retinitis pigmentosa becomes clinically evident. Both mouse and human data indicate that reactive retinal neovascularization either fails to develop or regresses when the number of photoreceptor cells is markedly reduced. The results show that a functional mechanism underlying this anti-angiogenic state is failure of the predicted up-regulation of vascular endothelial growth factor (VEGF), although other growth factors may also be involved. Preventive and therapeutic methods useful against both proliferative and degenerative retinopathies are also disclosed.

Ischemia-induced neovascularization of the retina is abolished in a mouse strain disclosed herein with inherited photoreceptor cell degeneration. Regression of established reactive retinal neovascularization caused by diabetes mellitus occurs in a subset of adult patients also afflicted with retinitis pigmentosa. This striking, previously unreported failure to mount a reactive retinal neovascularization response to potent exogenous stimuli is associated with an absence of the expected VEGF up-regulation in the retina. The results show that O₂ consumption by rod cells is a major driving force in ischemic retinal neovascularization that controls VEGF production. Additional trophic agents and cytokines are likely also to be involved in this complex biological phenomenon. Characterization of this anti-angiogenic state in the retina provides therapeutic approaches against important eye diseases including ischemic retinopathies and late complications of retinitis pigmentosa.

In certain embodiments of the invention, methods of reducing and/or preventing retinopathy of prematurity, diabetic retinopathy and/or retinal neovascularization may comprise exposing a neonate, a diabetic or another individual at risk for retinopathy to increased light. In some embodiments, light administration may utilize an apparatus designed to shine light into the eyes of the affected individual. In other embodiments, an apparatus designed to shine light on the closed eyelids of an affected individual may be used. However, the skilled artisan will realize that the invention is not limited to the use of apparatus for individualized light exposure, but may also utilize common light sources such as fluorescent overhead light fixtures and any other light emitting apparatus known in the art.

In various embodiments of the invention, an increase in light exposure may comprise an increase in light intensity and/or an increase in the duration of light exposure. In certain embodiments, low levels of light exposure during normal dark cycles (e.g., night time) may be preferred to prevent rod cells from becoming completely dark adapted. In alternative embodiments, an increase in light may represent an increased exposure to light relative to standard treatment protocols. For example, premature infants are typically exposed to reduced light levels in an attempt to decrease retinopathy of prematurity. In this case, light exposure may be increased relative to the reduced light exposure that is a present standard treatment for premature infants.

In other embodiments of the invention, increased light exposure may be supplemented with or replaced by other therapeutic agents, such as anti-angiogenic agents, rod cell metabolic inhibitors, VEGF inhibitors, anti-sense agents, cytotoxic agents, angiostatic agents and/or retinoic acid or vitamin A.

In some embodiments of the invention, novel inhibitors of retinal neovascularization may be identified by exposing selected mouse strains to hyperoxia, treating the mice with a putative inhibitor of retinal neovascularizaiton and assaying for inhibition of retinal neovascularization. Inhibition of retinal neovascularization may be determined using in vivo or in vitro assays.

### DETAILED DESCRIPTION OF THE INVENTION

Before the present methods, treatments and models are described, it is to be understood that this invention is not limited to the particular described embodiments and, as such, may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the administration” includes reference to one or more administrations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly humans, and includes:

(a) preventing the disease from occurring in a subject which may be predisposed to the disease but has not yet been diagnosed as having it;

(b) inhibiting the disease, i.e., arresting its development;

(c) decreasing the severity of the disease; or

(d) relieving the disease, i.e., causing regression of the disease.

The present invention offers new insight into the mechanism for and methods for the prevention of retinal neovascularization. The study utilizes two well-established mouse models of disease. In the first, a mouse model of oxygen-induced ischemic retinopathy, mice are exposed to 75% oxygen (O₂) from postnatal day 7 (P7) to P12, after which time they are returned to room air. Their retinas are analyzed 5-9 days later (P17-P21) by which time neovascularization has supervened on the retinal surface (Smith et al., Invest. Ophthalmol., 35:101-111, 1994). The exposure of neonatal mice to 75% O₂ causes vasoconstriction of the central retinal blood vessels. The decreased retinal perfusion, along with the return of the mice to room air, results in a relative retinal tissue hypoxia and ischemia, resulting in marked retinal neovascularization (Smith et al., Invest. Ophthalmol., 35:101-111, 1994; Pierce et al., Proc. Natl. Acad. Sci. USA, 92:905-909, 1995; Alon et al., Nat. Med., 1:1024-1028, 1995).

The mechanism underlying neovascularization in this animal model and in certain human diseases is thought to involve, among other factors, a hypoxia-driven up-regulation of VEGF (Pierce et al., Proc. Natl. Acad. Sci. USA, 92:905-909, 1995; Alon et al., Nat. Med., 1:1024-1028, 1995; Stone et al., J. Neurosci., 15:4738-4747, 1995; Duh et al., Diabetes, 48:1899-1906, 1997; Okamoto et al., Am. J. Pathol., 151:281-291, 1997; Aiello et al., Proc. Natl. Acad. Sci. USA, 92:10457-10461, 1995; Pierce et al., Arch. Ophthalmol., 114:1219-1228 1996). Over-expression of VEGF in the retina is sufficient to cause intraretinal and subretinal neovascularization (Okamoto et al., Am. J. Pathol., 151:281-291, 1997), whereas inhibition of VEGF expression or activity inhibits retinal neovascularization (Aiello et al., Proc. Natl. Acad. Sci. USA, 92:10457-10461, 1995).

VEGF, a 45 kDa glycoprotein that binds to several transmembrane tyrosine kinase receptors, is produced by glial cells of the neural retina, such as specialized astrocytes, including Muller cells among other cell types (Pierce et al., Proc. Natl. Acad. Sci. USA, 92:905-909, 1995; Alon et al., Nat. Med., 1:1024-1028, 1995; Stone et al., J. Neurosci., 15:47384747, 1995). VEGF expression in the retina decreases within 6 hours of exposure to 75% oxygen and remains decreased for the duration of the hyperoxia. In contrast, an increase in retinal VEGF expression is observed between 6 and 12 hours after the return to room air, and such expression remains elevated during development of the neovascularization. Therefore, VEGF levels play a dual role in this retinopathy model: a down-regulation of VEGF by hyperoxia induces blood vessel regression, while subsequent up-regulation of VEGF leads to retinal neovascularization (Alon et al., Nat. Med., 1:1024-1028, 1995; Pierce et al., Arch. Ophthalmol., 114:1219-1228, 1996).

The second mouse model used in the present invention is the classic autosomal recessive inherited degenerative disease of photoreceptor cells known as retinal degeneration, Pdeb^{dr1}. This disease is caused by a nonsense mutation in the beta subunit of the rod photoreceptor cell-specific phosphodiesterase (Sidman et al., J. Hered., 56, 23-29, 1965; Bowes et al., Nature, 347:677-680, 1990; Pittler et al., Proc. Natl. Acad. Sci. USA, 88:8322-8326, 1991; Lem et al., Proc. Natl. Acad. Sci. USA, 89:4422-4426, 1992). Light absorption by rhodopsin activates transducin, a G-protein, which in turn promotes cGMP hydrolysis by the specific phosphodiesterase, leading to hyperpolarization of rod photoreceptor cells (Roof et al., Principles and Practice of Ophthalmology, W. B. Saunders Company, Philadelphia, 2000).

The widely distributed Pdeb^{rd1} mutation (Sidman et al., J. Hered., 56, 23-29, 1965) can be traced back directly to Keeler's rodless mutation (Keeler et al., Proc. Natl. Acad. Sci. USA, 10:329-333, 1924), as shown by analysis of DNA extracted from Keeler's original microscope slides 70 years later (Pittler et al., Proc. Natl. Acad. Sci. USA, 90:9616-9619, 1993). The retinal development in Pdeb^{rd1}/Pdeb^{rd1} mice proceeds normally until Pl1. At that time development of photoreceptor cell outer segments arrests and the rod cell nuclei, inner segments and outer segments begin to degenerate. Photoreceptor cell degeneration then proceeds rapidly, and exceeds 80% by P15, and 90% by P21 (Farber, D. B., Invest. Ophthalmol. Vis. Sci, 36:263-275, 1995). By P25-30 only one sparsely populated row of photoreceptor cell nuclei remains and the outer segments have disappeared. By the beginning of the fourth postnatal week, most surviving photoreceptor cells are cone cells (Carter-Dawson et al., Invest. Ophthalmol. Vis. Sci., 17:489498, 1978; IaVail et al., Exp. Eye Res., 23:227-245, 1976).

Apoptosis of the photoreceptor cell is the final pathogenic event common to all animal models of retinal degeneration (Chang et al., Neuron, 11:595-605, 1993; Portera-Cailliau et al., Proc. Natl. Acad. Sci. USA, 91:974-978, 1994). In addition to the primary photoreceptor cell loss, Pdeb^{rd1} mutant mice (Blanks et al., J. Comp. Neurol., 254:543-553, 1986) and patients with retinitis pigmentosa (Grunwald et al., Am. J. Ophthamol., 122:502-508, 1996) may also have an altered retinal blood flow.

### BRIEF DESCRIPTIONS OF THE DRAWINGS

FIG. 1a. Effect of relative hypoxia on C57BL/6+/+wt and on Pdeb^{rd1}/Pdeb^{rd1} mutant mouse retinas. Wt retina on P17 of a mouse kept continuously in room air. Scale bar; 100 μm (a-d) and 35 μm (e-h). ONL; outer nuclear layer.

FIG. 1b. Effect of relative hypoxia on C57BL/6+/+wt and on Pdeb^{rd1}/Pdeb^{rd1} mutant mouse retinas. Wt retina on P17 after exposure to 75% oxygen for 5 days from P7 to P12. A large number of new blood vessels is seen protruding into the vitreous space. Arrows point to endothelial cell nuclei. Scale bar; 100 μm (a-d) and 35 μm (e-h). ONL; outer nuclear layer.

FIG. 1c. Effect of relative hypoxia on C57BL/6+/+wt and on Pdeb^{rd1}/Pdeb^{rd1} mutant mouse retinas. Pdeb^{rd1}/Pdeb^{rd1} retina on P17 when mouse has been kept in room air. Scale bar; 100 μm (a-d) and 35 μm (e-h). ONL; outer nuclear layer.

FIG. 1d. Effect of relative hypoxia on C57BL/6+/+wt and on Pdeb^{rd1}/Pdeb^{rd1} mutant mouse retinas. Pdeb^{rd1}/Pdeb^{rd1} retina on P17 after exposure to 75% oxygen for 5 days from P7 to P12. No new blood vessels are seen protruding into the vitreous. Scale bar; 100 μm (a-d) and 35 μm (e-h). ONL; outer nuclear layer.

FIG. 1e. Effect of relative hypoxia on C57BL/6+/+wt and on Pdeb^{rd1}/Pdeb^{rd1} mutant mouse retinas. Anti-von Willebrand (vWF) factor antibody-immunostained section of wt retina on P17 after exposure to 75% oxygen from P7 to P12. Arrows point to endothelial cell nuclei. Scale bar; 100 μm (a-d) and 35 μm (e-h).

FIG. 1f. Effect of relative hypoxia on C57BL/6+/+wt and on Pdeb^{rd1}/Pdeb^{rd1} mutant mouse retinas. Detail of a hematoxylin and eosin (H&E)-stained section of a typical new blood vessel in a wt retina after exposure to 75% oxygen from P7 to P12. Arrows point to endothelial cell nuclei. Scale bar; 100 μm (a-d) and 35 μm (e-h).

FIG. 1g. Effect of relative hypoxia on C57BL/6+/+wt and on Pdeb^{rd1}/Pdeb^{rd1} mutant mouse retinas. vWF-antibody-stained section of Pdeb^{rd1}/Pdeb^{rd1} retina on P17 after exposure to 75% oxygen from P7 to P12Scale bar; 100 μm (a-d) and 35 μm (e-h).

FIG. 1 h. Effect of relative hypoxia on C57BL/6+/+wt and on Pdeb^{rd1}/Pdeb^{rd1} mutant mouse retinas. Detail of a H&E-stained section of a Pdeb^{rd1}/Pdeb^{rd1} retina on P17 after exposure to 75% oxygen from P7 to P12. Scale bar; 100 μm (a-d) and 35 μm (e-h).

FIG. 2a. VEGF expression in wt mouse and Pdeb^{rd1}/Pdeb^{rd1} mouse retinas. Northern blot analysis of VEGF expression in wt (lanes 1-3) and Pdeb^{rd1}/Pdeb^{rd1} (lanes 4-6) mouse retina. Wild-type and Pdeb^{rd1}/Pdeb^{rd1} mice were exposed to 75% oxygen from P7 to P12. On P12, retinal RNA was isolated immediately (0 h; lanes 2 and 5) or 12 hours (12 h; lanes 3 and 6) after return to room air from 75% oxygen. Retinal VEGF expression was quantified also from retinas of mice kept only in room air until P12 (ctrl; lanes 1 and 4). Arrow indicates VEGF transcript (3800 bp).

FIG. 2b. VEGF expression in wt mouse and Pdeb^{rd1}/Pdeb^{rd1} mouse retinas. 28S and 18S ribosomal markers serve as loading controls.

FIG. 2c. Integrated density values of VEGF transcripts shown in FIG. 2a and FIG. 2b were quantified. The baseline value for VEGF expression in wt mice kept only in room air until P12 was set to 1.0. Standard deviations were typically less than 10% of the mean. A representative experiment is shown.

FIG. 3. Spontaneously regressed optic disc neovascularization (arrow) in a 36-year-old woman with concurrent type I diabetes mellitus and retinitis pigmentosa. Note the granular and “bone spicule-like” pigmentary changes in the retina (asterisks) consistent with a diagnosis of retinitis pigmentosa.

### ROD CELL METABOLIC INHIBITORS

The present invention contemplates the use of rod cell metabolic inhibitors for therapeutic treatment of retinopathies. In certain embodiments, known inhibitors of rod cell metabolism may be used. These fall into general categories, such as inhibitors of mitochondrial function (e.g., rotenone, amytal, antimycin A, oligomycin); cGMP antagonists or kinase inhibitors (e.g., H-8, Rp-8-bromo-cGMP, Rp-8-pCPI-cGMPS, Rp-8-Br-cGMPS); and anesthetics (e.g., barbiturates, lidocaine, procaine, etc.). In alternative embodiments, targeted cytotoxic agents may also be used to prevent or inhibit retinopathies and/or retinal neovascularization. Any cytotoxic agent known in the art, such as pro-apoptosis agents (gramicidin, magainin, mellitin, defensin, cecropin, (KLAKLAK)₂, (KLAKKLA)₂, (KAAKKAA)₂ or (KLGKKLG)₃) may be used within the scope of the invention. In preferred embodiments, such inhibitors or cytotoxic agents may be selectively targeted to retinal vasculature by cross-linking to retinal selective targeting peptides (see, e.g., PCT patent application serial No. PCT/US01/27692, filed Sep. 7, 2001, the entire text of which is incorporated herein by reference).

In some embodiments of the invention, assays may be performed for novel rod cell metabolic inhibitors. Assays for rod cell metabolic inhibitors may make use of a variety of different formats and may depend on the kind of “activity” for which the screen is being conducted. Contemplated functional “read-outs” include oxygen consumption rates, cGMP phosphodiesterase activity, Na+/Ca++ channel activity, electrical (synaptic) activity, ATP/ADP levels, glycolytic enzyme activity, intracellular pH, glucose consumption rates, etc. In preferred embodiments, inhibitors are directed to oxidative rod cell metabolism, which can be assayed, for example, by oxygen consumption rates. However, any known metabolic assay may be used within the scope of the invention.

In Vitro Assays

In certain embodiments, screening of compounds as metabolic inhibitors of rod cells may utilize in vitro assays. For example, particular enzymatic targets may be screened for inhibitor binding and/or inhibition of catalytic activity. Activity assays for different types of proteins are well known in the art and any such known assay may be used. For example, oxygen consumption assays using isolated mitochondria are well known, as are assays for various components of the mitochondrial electron transfer chain.

Various transport proteins, such as Na+/Ca++ transporters, may be assayed in artificial lipid bilayer systems or using isolated membranes in combination with electrical conductance measurements or ion-specific electrodes. In vitro assays for phosphodiesterase activity are well known. Binding activity may be measured by, for example, by exposing radiolabeled or other tagged molecules to target proteins that have been covalently or non-covalently attached to a surface and detecting the presence of tagged molecules bound to the surface. Any such known method may be used within the scope of the present invention.

Various cell lines containing wild-type or natural or engineered mutations in rod cells can be used to study various functional attributes of potential inhibitors. Methods for engineering mutations are well known in the art. In such assays, a test compound would be formulated appropriately, given its biochemical nature, and contacted with a target cell. Depending on the assay, culture may be required. The cell may then be examined by virtue of a number of different physiologic assays, such as those listed above. Alternatively, molecular analysis may be performed in which the function of rod cells may be explored. This may involve assays such as those for oxygen consumption, cGMP levels, ion transport activity, or any other process related to rod cell metabolism.

In Vivo Assays

The present invention also encompasses the use of various animal models, such as the C57BL/6+/+wt and on Pdeb^{rd1}/Pdeb^{rd1} mutant mice strains discussed herein. The effect of various putative rod cell inhibitors on rod cell oxidative metabolism and/or neovascularization may be examined as disclosed in the following Examples.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal, intravenous or intraarterial injection.

Determining the effectiveness of a compound in vivo may involve a variety of different criteria. Such criteria include, but are not limited to: rod cell oxidative metabolism, neovascularization, protrusion of vascular endothelial cell nuclei into the vitreous space and/or VEGF expression.

Rational Drug Design

The goal of rational drug design is to produce structural analogs of biologically active compounds (agonists, antagonists, inhibitors, binding partners, etc.). By creating such analogs, it is possible to fashion drugs that are more active or stable than the natural molecules, which have different susceptibility to alteration or which may affect the function of various other molecules. In one approach, one would generate a three-dimensional structure for rod cell protein or a fragment thereof. This could be accomplished by x-ray crystallography, computer modeling based on the 3-D structures of other similar proteins or by a combination of both approaches. In addition, knowledge of the polypeptide sequences permits computer employed predictions of structure-function relationships. An alternative approach, an “alanine scan,” involves the random replacement of residues throughout a protein or peptide molecule with alanine, followed by determining the resulting effect(s) on protein function.

It also is possible to isolate a rod cell specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of an anti-idiotype antibody would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

cGMP Antagonists

A potential class of inhibitors that may be of use to inhibit rod cell oxidative metabolism consists of cGMP antagonists, such as H-8 (Wei et al., Neurosci. Lett. 237:3740, 1997), Rp-8-bromo-cGMP, Rp-8-pCPT-cGMPS and Rp-8-Br-cGMPS.

Since ion transport activity and neurotransmitter release by rod cells is dependent on cGMP, administration of cGMP antagonists may act to inhibit rod cell oxidative metabolism. Such inhibitors are well known in the art and many examples of cGMP antagonists are commercially available. Rational drug design may be used to enhance the efficacy, lifetime or selectivity of cGMP antagonists and/or other types of rod cell metabolic inhibitors.

Anti-Angiogenic Factors

A further embodiment of the present invention concerns the use of antiangiogenic agents and/or gene therapy as adjuncts for therapeutic treatment of retinopathies. Antiantiogenic gene therapy may be accomplished, for example, by the methods of Lin et al. (Proc. Natl. Acad. Sci. USA, 95: 8829-8834, 1998). Alternatively, antiangiogenic agents, such as AGM-1470 (TNP470), platelet factor 4 and/or angiostatin may be used as adjuncts for treatment of retinopathies (Folkman, In: The Molecular Basis of Cancer(Mendelsohn et al., eds.), pp. 206-232, W B Saunders, Philadelphia, 1995). Additional antiangiogenic agents that may be used in the practice of the present invention are identified in Augustin (Trends Pharmacol. Sci., 19: 216-222, 1998).

Antisense and Ribozyme Constructs

Antisense

Certain embodiments of the invention may involve therapeutic use of antisense constructs. The term “antisense” refers to polynucleotide molecules complementary to a portion of a targeted gene or mRNA species. Complementary polynucleotides are those that are capable of base-pairing according to the standard Watson-Crick complementarity rules. That is, purines will base pair with pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with base pairing.

Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

The intracellular concentration of monovalent cations is approximately 160 mM (10 mM Na⁺; 150 mM K⁺). The intracellular concentration of divalent cations is approximately 20 mM (18 mM Mg⁺; 2 mM Ca++). The intracellular protein concentration, which would serve to decrease the volume of hybridization and, therefore, increase the effective concentration of nucleic acid species, is 150 mg/ml. Constructs may be tested for specific hybridization in vitro under conditions that mimic these in vivo conditions.

Antisense constructs may be designed to bind to the promoter and other control regions, exons, introns or even exon-intron boundaries of a gene. In certain embodiments, it is contemplated that effective antisense constructs may include regions complementary to the mRNA start site. In preferred embodiments, the antisense constructs are targeted to a sequence of an hnRNA and/or mRNA that is present in VEGF (GenBank Accession Numbers AF_{—}095,785; XM_{—}166,457; NM_{—}003,376). For example, one might target the promoter or the 5′ end of the mRNA encoding VEGF (AF_{—}095,785). One of ordinary skill in the art can readily test such constructs to determine whether levels of the target protein are affected. In some embodiments, anti-sense contructs against VEGF may be administered to individuals at risk for retinopathy of prematurity, diabetic retinopathy and/or retinal neovascularization as an adjunct to or in place of light based therapy.

As used herein, the terms “complementary” or “antisense” mean polynucleotides that are substantially complementary to the target sequence over their entire length and have very few base mismatches. For example, sequences of fifteen bases in length may be termed complementary when they have a complementary nucleotide at thirteen or fourteen nucleotides out of fifteen. Naturally, sequences that are “completely complementary” will be sequences that are entirely complementary throughout their entire length and have no base mismatches. Other sequences with lower degrees of homology also are contemplated. For example, an antisense construct that has limited regions of high homology, but also contains a non-homologous region (e.g., a ribozyme) could be designed. These molecules, though having less than 50% homology, would bind to target sequences under appropriate conditions.

Although the antisense sequences may be full length cDNA copies, or large fragments thereof, they also may be shorter fragments, or “oligonucleotides,” defined herein as polynucleotides of 50 or less bases. Although shorter oligomers (8-20) are easier to make and increase in vivo accessibility, numerous other factors are involved in determining the specificity of base-pairing. For example, both binding affinity and sequence specificity of an oligonucleotide to its complementary target increase with increasing length. It is contemplated that oligonucleotides of 8, 9, 10, 11, 12, 13, 14, 15, 16, 17, 18, 19, 20, 25, 30, 35, 40, 45, 50 or 100 base pairs will be used. While all or part of the gene sequence may be employed in the context of antisense construction, statistically, any sequence of 14 bases long should occur only once in the human genome and, therefore, suffice to specify a unique target sequence.

In certain embodiments, one may wish to employ antisense constructs which include other elements, for example, those which include C-5 propyne pyrimidines. Oligonucleotides which contain C-5 propyne analogues of uridine and cytidine have been shown to bind RNA with high affinity and to be potent antisense inhibitors of gene expression (Wagner et al., Science, 260:1510-1513, 1993).

Alternatively, the antisense oligo- and polynucleotides according to the present invention may be provided as RNA via transcription from expression constructs that carry nucleic acids encoding the oligo- or polynucleotides. Throughout this application, the term “expression construct” is meant to include any type of genetic construct containing a nucleic acid encoding a product in which part or all of the nucleic acid sequence is capable of being transcribed. Typical expression vectors include bacterial plasmids or phage, such as any of the pUC or Bluescript™plasmid series or viral vectors adapted for use in eukaryotic cells.

In preferred embodiments, the nucleic acid encodes an antisense oligo- or polynucleotide under transcriptional control of a promoter. A “promoter” refers to a DNA sequence recognized by an RNA polymerase to initiate the specific transcription of a gene. The phrase “under transcriptional control” means that the promoter is in the correct location and orientation in relation to the nucleic acid to control RNA polymerase initiation.

The term promoter will be used here to refer to a group of transcriptional control modules that are clustered around the initiation site for RNA polymerase II. Promoters are composed of discrete functional modules, each consisting of approximately 7-20 bp of DNA, and containing one or more recognition sites for transcriptional activator or repressor proteins. At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid encoding the inhibitory polynucleotide is not believed to be important, so long as it is capable of expressing the peptide in the targeted cell. Thus, where a human cell is targeted, it is preferable to position the nucleic acid coding the inhibitory peptide adjacent to and under the control of a promoter that is active in the human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used to obtain high-level transcription. The use of other viral or mammalian cellular or bacterial phage promoters which are well-known in the art is contemplated as well, provided that the levels of transcription and/or translation are sufficient for a given purpose.

Selection of a promoter that is regulated in response to specific physiologic signals can permit inducible expression of an antisense sequence. For example, a nucleic acid under control of the human PAI-I promoter results in expression inducible by tumor necrosis factor. Additionally any promoter/enhancer combination also could be used to drive expression of a nucleic acid according to the present invention. Tables 1 and 2 list elements/promoters that may be employed to regulate transcription and/or translation of operably coupled genes. This list is exemplary only and any known promoter and/or regulatory element may be used.

TABLE 1
ENHANCER/PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Prealbumin (Transthyretin)
Muscle Creatine Kinase
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus

TABLE 2

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X, poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2 kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

Ribozymes

Another method for inhibiting the expression of VEGF or other gene products is via ribozymes. Ribozymes are RNA-protein complexes that cleave nucleic acids in a site-specific fashion. Ribozymes have specific catalytic domains that possess endonuclease activity (Kim and Cech, Proc. Natl. Acad. Sci. USA, 84:8788-8792, 1987). For example, a large number of ribozymes accelerate phosphoester transfer reactions with a high degree of specificity, often cleaving only one of several phosphoesters in an oligonucleotide substrate (Cech et al., Cell, 27:487496, 1981). This specificity has been attributed to the requirement that the substrate bind via specific base-pairing interactions to an internal guide sequence (“IGS”) of the ribozyme prior to chemical reaction.

Ribozyme catalysis has primarily been observed as part of sequence-specific cleavage/ligation reactions involving nucleic acids (Joyce, Nature, 338:217-244, 1989; Cech et al., 1981). For example, U.S. Pat. No. 5,354,855 reports that certain ribozymes can act as endonucleases with a sequence specificity greater than that of known ribonucleases. Thus, sequence-specific ribozyme-mediated inhibition of gene expression may be particularly suited to therapeutic applications (Scanlon et al., Proc Natl Acad Sci USA, 88:10591-10595, 1991; Sarver et al., Science, 247:1222-1225, 1990; Sioud et al., J. Mol. Biol., 223:831-835, 1992). It was reported that ribozymes elicited genetic changes in some cells lines to which they were applied.

Several different ribozyme motifs have been described with RNA cleavage activity (Symons, Annu. Rev. Biochem., 61:641-671, 1992). Examples that may function equivalently include sequences from the Group I self splicing introns including Tobacco Ringspot Virus (Prody et al., Science, 231:1577-1580, 1986), Avocado Sunblotch Viroid (Palukaitis et al., Virology, 99:145-151, 1979; Symons, Nucl. Acids Res., 9:6527-6537), and Lucerne Transient Streak Virus (Forster and Symons, Cell, 49:211-220, 1987). Sequences from these and related viruses are referred to as hammerhead ribozymes. Other suitable ribozymes include sequences from RNase P (Yuan et al., Proc. Natl. Acad. Sci. USA, 89:8006-8010, 1992, Yuan and Altman, Science, 263:1269-1273, 1994, U.S. Pat. Nos. 5,168,053 and 5,624,824), hairpin ribozyme structures (Berzal-Herranz et al., Genes and Devel., 6:129-134, 1992; Chowrira et al., Biochemistry, 32:1088-1095, 1993) and Hepatitis Delta virus based ribozymes (U.S. Pat. No. 5,625,047). The general design and optimization of ribozyme directed RNA cleavage activity has been discussed in detail (Haseloff and Gerlach, Nature, 334:585-591, 1988, Symons, 1992, Chowrira et al., “In vitro and in vivo comparison of hammerhead, hairpin, and hepatitis delta virus self-processing ribozyme cassetyes,” J. Biol. Chem., 269:25856-25864, 1994).

The other variable in ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence that is the cleavage site. For hammerhead ribozymes, the cleavage site is a dinucleotide sequence on the target RNA—a uracil (U) followed by either an adenine, cytosine or uracil (A,C orU) (Perriman et al., Gene, 113:157-163, 1992).

The large number of possible cleavage sites in genes of moderate size, coupled with the growing number of sequences with demonstrated catalytic RNA cleavage activity indicates that a large number of ribozymes that have the potential to downregulate gene expression are available. Additionally, due to the sequence variation among different genes, ribozymes could be designed to specifically cleave individual genes or gene products. Designing and testing ribozymes for efficient cleavage of a target RNA is a process well known to those skilled in the art. Examples of scientific methods for designing and testing ribozymes are described by Chowrira et al., (1994).

Expression Vectors

Nucleic acids encoding anti-VEGF antisense constructs may be incorporated into expression vectors. The nucleic acid encoding an antisense construct may be inserted into an expression vector by standard subcloning techniques. The engineering of DNA segment(s) for expression in eukaryotic system may be performed by techniques generally known to those of skill in the art. It is believed that virtually any expression system may be employed in the expression of the antisense constructs.

To express an antisense construct in accordance with the present invention one would prepare an expression vector that comprises a sequence encoding, for example, anti-VEGF under the control of, or operatively linked to, one or more promoters. One positions the 5′ end of the transcription initiation site of the transcriptional reading frame generally between about 1 and about 50 nucleotides “downstream” (ie., 3′) of the chosen promoter. The “upstream” promoter stimulates transcription of the DNA.

The promoters may be derived from the genome of mammalian cells (e.g., metallothionein promoter) or from mammalian viruses (e.g., the adenovirus late promoter; the vaccinia virus 7.5K promoter). A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment that also contains the SV40 viral origin of replication. Smaller or larger SV40 fragments may also be used, provided there is included the approximately 250 bp sequence extending from the Hind m site toward the Bgl I site located in the viral origin of replication.

Formulations and Routes for Administration to Patients

In certain embodiments, inhibitors of rod cell oxidative metabolism and/or other therapeutic agents, such as anti-angiongenic agents or pro-apoptosis agents, may be used for therapeutic treatment of retinopathies. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

Aqueous compositions of the present invention comprise an effective amount of a therapeutic agent, dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the rod cell inhibitors or activators of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions normally would be administered as pharmaceutically acceptable compositions.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present invention may be formulated in a neutral or salt form. Pharmaceutically-acceptable salts include the acid addition salts which are formed by reaction of basic groups with inorganic acids such as, for example, hydrochloric or phosphoric acids, or such organic acids as acetic, oxalic, tartaric, mandelic, and the like. Salts formed with free acidic groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the studies below are all or the only studies performed. Efforts have been made to ensure accuracy with respect to numbers used (e.g. amounts, temperature, etc.) but some errors and deviations may occur. Unless indicated otherwise, parts are parts by weight, molecular weight is weight average molecular weight, temperature is in degrees Centigrade, and pressure is at or near atmospheric.

### Example 1

### Rod Cell Oxidative Metabolism and Retinopathy

Materials and Methods

Animals. This study adhered to the Association for Research in Vision and Ophthalmology (ARVO) Statement for the Use of Animals in Ophthalmic and Vision Research. The strain of inbred congenic C57BL/6 mice carrying the Pdeb^{rd1} mutation has been described (LaVail et al., Vision Res., 14:693-702, 1974). The disclosed studies used mice at the F77N2F14-16 generations. C57BL/6+/+wt mice (Harlan, Indianapolis, Ind.) were used as controls.

Induction of retinal neovascularization. P7 mouse pups with their nursing mothers were exposed to 75% oxygen for 5 days. Mice were returned to room air (20.8% O₂) on P12. For histological analysis mice were sacrificed between P17 and P21 and eyes were enucleated and fixed in 4% paraformaldehyde in PBS overnight at 4° C. For RNA isolation, mice were sacrificed and their eyes were enucleated on P12 either immediately or 12 hours after return to room air from 75% O₂. Retinas were dissected and stored in TR1-reagent (Sigma, St. Louis, Mo.) at −80° C.

Histological and immunohistochemical analysis. Fixed and alcohol dehydrated eyes were embedded in paraffin and serially sectioned at 5 μm. Tissue sections were stained either with hematoxylin and eosin (H&E) or immunostained with an anti-von Willebrand (vWF) factor antibody (DAKO, Carpinteria, Calif.) according to the manufacturer's instructions. Endothelial cell nuclei on the vitreous side of the internal limiting membrane were counted (Smith et al., Invest. Ophthalmol. Vis. Sci, 35:101-111, 1994). At least six H&E-stained sections were evaluated per eye, and the average number of nuclei was counted from at least eight eyes for each experimental condition. Student's t-test was used to determine whether the differences observed were statistically significant. All studies were repeated at least three times under similar conditions.

Northern blot analysis and in situ hybridization. RNA was isolated from mouse retinas using TR1-reagent (Sigma) according to the manufacturer's instructions. Total retinal tissue RNA (8 μg per sample) from each time point was electrophoresed on a 1% agarose gel containing 6% formaldehyde (Cheng et al., Proc. Natl. Acad. Sci USA 93:8502-8507, 1996). RNA was transferred to nylon membranes and hybridized with a ³²P-labelled VEGF₁₆₅ cDNA probe (Cheng et al., Proc. Natl. Acad. Sci. USA 93:8502-8507, 1996). Densitometry data were acquired and analyzed by using a Fluor Chem imager and software (Alpha Innotech Corporation, San leandro, CA). Colorimetric in situ hybridization of paraffin-embedded eyes was performed with hyperbiotinylated oligoprobes (Kitadai et al., Clin. Cancer Res., 1:1095-1102, 1995).

RESULTS

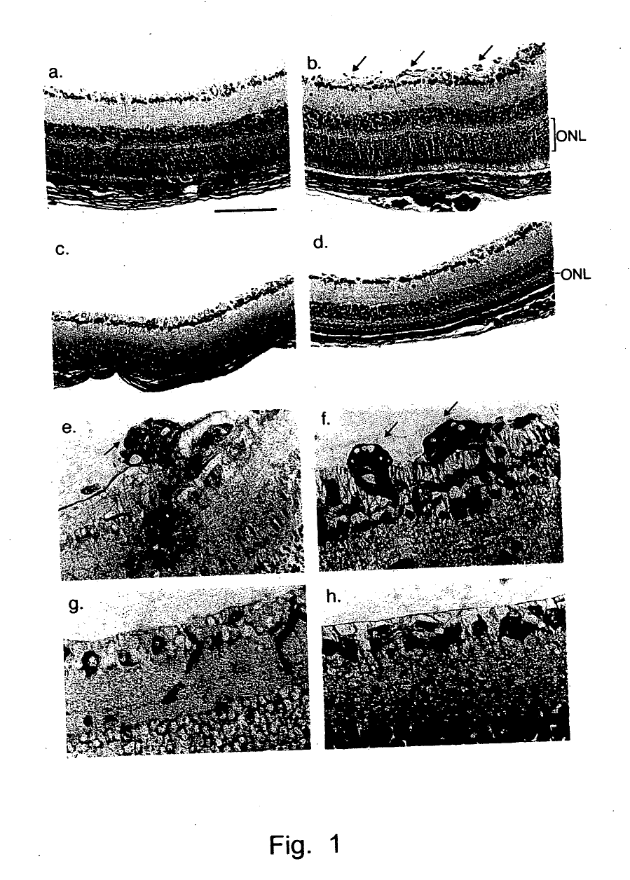

Abolishment of reactive retinal neovascularization in young mice with inherited retinal degeneration. To test the angiogenic response of the Pdeb^{rd1} mutant retinas in response to ischemia, simultaneous studies were designed with the mouse models of O₂-induced retinopathy and retinal degeneration. Combination of the models produced the surprising finding that the reactive retinal neovascularization characteristic of normal young mice exposed to high O₂ levels, and observed in wild-type (wt) and heterozygous animals, failed to occur in Pdeb^{rd1} homozygotes. Neovascularization was quantified by counting vascular endothelial cell nuclei protruding into the vitreous space from at least six sections of 8-36 eyes in five independent experiments (Table 1). Results of these studies are also illustrated in FIG. 1.

TABLE 1
Effect of O₂-induced retinal neovascularization
in wild-type and Pdeb^{rd1}/Pdeb^{rd1} mice.*

| | Eyes with retinal | |
|---|---|---|
| | neovascularization/ | |
| | Total number of eyes examined^{#} | |
| Condition/Genotype | +/+ | Pdeb^{rd1}/Pdeb^{rd1} |
| Control (Room Air, P1-P17) | 0/8 | 0/12 |
| Experimental (75% O_{2,} P7-P12) | 36/36 | 0/34⁺ |

*Results were compiled from five independent studies.
^{#}Retinas were examined on P17.
⁺p < 0.0001

Extensive induction of retinal neovascularization (40.0±3.2 endothelial cell nuclei/eye section) was seen in C57BL/6+/+wt mice on P17 after 75% oxygen treatment from P7 to P12 (FIG. 1b) and in heterozygous+/Pdeb^{rd1} mice. Virtually no endothelial cell nuclei (0.4±0.1 endothelial cell nuclei/eye section) were seen in the Pdeb^{rd1}/Pdeb^{rd1} retinas on P17 after exposure to 75% oxygen from P7 to P12 (FIG. 1d). At this time only a few layers of nuclei remained in the photoreceptor cell layer. Also, no endothelial cell nuclei were seen on or after P21, ruling out the possibility of delayed retinal neovascularization (data not shown). No endothelial cell nuclei were seen on P17 in either wt or Pdeb^{rd1}/Pdeb^{rd1} mice exposed only to room air (FIG. 1a and FIG. 1c). Staining for von Willebrandt Factor confirmed that the cells protruding into the vitreous of wt mice treated with 75% oxygen were indeed endothelial cells (FIG. 1e and FIG. 1g) and that such cells were almost completely confined to the neural retina in Pdeb^{rd1}/Pdeb^{rd1} homozygotes (FIG. 1g and FIG. 1 h).

Failure of the predicted up-regulation of VEGF in mice with inherited retinal degeneration. VEGF has been suggested to be one of the key angiogenic factors in oxygen-induced retinal neovascularization (Pierce et al., Proc. Natl. Acad. Sci. USA, 92:905-909, 1995; Alon et al., Nat. Med., 1:1024-1028, 1995; Stone et al., J. Neurosci., 15:47384747, 1995; Duh et al., Diabetes, 48:1899-1906, 1997; Okamoto et al., Am. J.

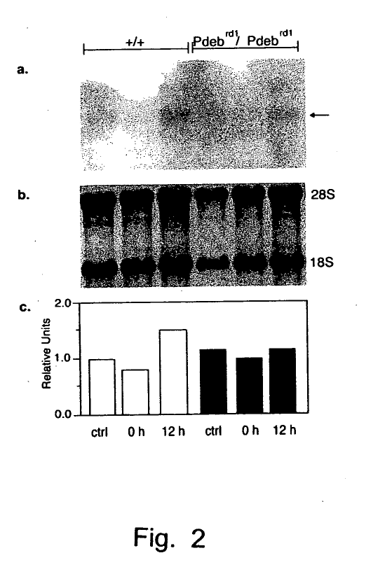

Pathol., 151:281-291, 1997; Aiello et al., Proc. Natl. Acad. Sci. USA, 92:10457-10461, 1995; Pierce et al., Arch. Ophthalmol., 114:1219-1228, 1996). Results provided here show that differences in VEGF expression play a role in the lack of neovascularization in the retinas of Pdeb^{rd1}/Pdeb^{rd1} mice and examined VEGF-expression in retinal tissue by Northern blot analysis (FIG. 2). Total RNAs from wt and Pdeb^{rd1}/Pdeb^{rd1} mouse retinas were analyzed on P12 after exposing mice for 5 days to either 75% O₂ or to room air. A decline in VEGF expression was seen during exposure to hyperoxia. This decrease was followed by a 150% increase in the VEGF expression in wt mouse retinas observed 12 hours after the return to room air after 75% O₂ exposure, compared to that seen following exposure to room air only. In Pdeb^{rd1}/Pdeb^{rd1} mice retinal VEGF expression remained low and unchanged even after exposure to 75% O₂ for 5 days, comparable to retinas of similar (otherwise isogenic) mice exposed only to room air.

To determine whether inhibition of neovascularization was a consequence of an altered spatial expression pattern of VEGF rather than an overall alteration in VEGF expression levels in the Pdeb^{rd1}/Pdeb^{rd1} mouse retina, VEGF-expression was analyzed in the retina by in situ hybridization. Tissue sections from wt and Pdeb^{rd1}/Pdeb^{rd1} mouse eyes were evaluated on P12, 12 hours after exposure to either 75% O₂ or room air for 5 days. Slightly higher VEGF mRNA levels were seen in the inner nuclear layer and in the inner plexiform layer of wt mouse retinas on P12, after 12 hours in room air following 75% O₂ exposure. These expression patterns are consistent with previous studies (Pierce et al., Proc. Natl. Acad. Sci. USA, 92:905-909, 1995), but a comparable increase in VEGF expression was not seen in any region in Pdeb^{rd1}/Pdeb^{rd1} mouse retinas after 75% O₂ exposure.

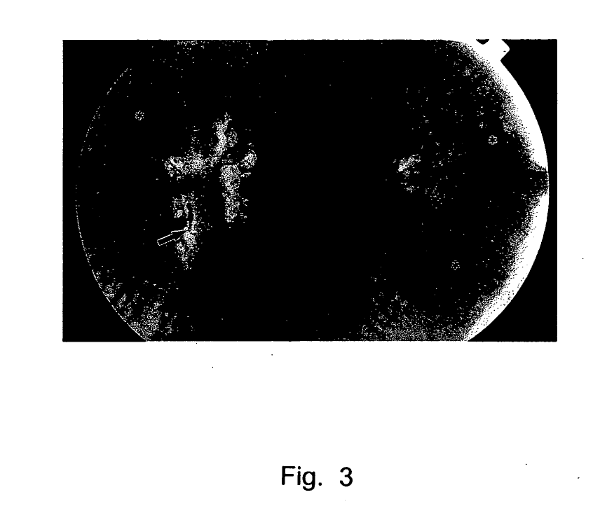

Regression of diabetic retinopathy in some patients with retinitis pigmentosa. There are clinical counterparts to the mouse studies described here in which an exogenous stimulus of pathological formation of new retinal blood vessels fails in the presence of advanced photoreceptor cell degeneration. A clinical case is described herein where proliferative retinopathy regressed spontaneously in a diabetic patient with concurrent retinitis pigmentosa. On fundus examination of a 36-year-old female, diagnosed with type I diabetes mellitus for the past 34 years, inactive fibroglial membranes projecting into the vitreous from the optic discs were observed in both eyes (FIG. 3). This pattern was consistent with regressed retinal neovascularization, often observed in cases of patients with proliferative diabetic retinopathy after successful laser treatment (Roof et al., Principles and Practice of Ophthalmology, W. B. Saunders Company, Philadelphia, 2000). However, the patient had never received laser treatment. In the periphery and midperiphery of the fundus, attenuated vasculature and atrophic retina with granular and bone spicule pigmentary changes were observed, consistent with a diagnosis of retinitis pigmentosa (FIG. 3) and confirmed by a virtually flat electroretinogram (i.e., less than 10 μV). In non-diabetics with retinitis pigmentosa, spontaneous regression of optic disc neovascularization caused by an unknown mechanism can also occur (Hayakawa et al., Am. J. Ophthalmol., 115:168-173, 1993). This example combined with other sporadic clinical case reports (Hayakawa et al., Am. J. Ophthalmol., 115:168-173, 1993; Uliss et al., Ophthalmology, 93:1599-1603, 1986; Butner, R. W. Ann. Ophthalmol., 16:861,863-865, 1984) indicates that in the clinical setting of rod photoreceptor cell degeneration, proliferative retinopathies may fail to develop or may regress early.

Discussion

The pathogenesis of neovascularization in ischemic retinopathies is best considered in the context of vasculogenesis in the normal developing retina. Blood vessels enter the back of the embryonic eye at the eye-cup stage and reach the vitreal surface via the choroidal fissure. This fissure closes around the developing optic nerve and the blood vessels close to the vitreal surface that supply the innermost part of the central retina. As the retina expands during and after the fetal period, the vessels branch and grow radially outward toward the retinal periphery. Astrocytes lie in the avascular zone just ahead of the radially spreading vessels, and are thought to stimulate and control the direction of vessel growth by local release of VEGF (Pierce et al., Proc. Natl. Acad. Sci USA,92:905-909, 1995; Alon et al., Nat. Med., 1:1024-1028, 1995; Stone et al., J. Neurosci., 15:47384747, 1995; Roof et al. Principles and Practice of OphthalmologyW. B. Saunders Company, Philadelphia, 2000; Schlingemann et al., Br. J. Ophthalmol., 81:501-512, 1997). Astrocytes also grow inward into the inner plexiform and inner nuclear layers of the developing retina, and stimulate the growth of immature vessels inward in their path.

Once blood vessels mature to the stage at which they are invested with peri-endothelial cells (“pericytes”) they lose responsiveness to VEGF (Benjamin et al., J. Clin. Invest., 103:159-165, 1999). Another set of blood vessels supplies the choroid, just external to the pigment epithelial layer. The pigment epithelial layer itself and the entire length of the photoreceptor cells, from their synaptic endings in the outer plexiform layer, through their nuclei in the outer nuclear layer, to their specialized inner and outer segments close to the pigment epithelium are normally avascular. In experimental or clinical contexts in which retinal hypoxia induces VEGF expression, new blood vessels will form either on the inner neural retina in young subjects or, in some older subjects, from the choroid across the pigment epithelium (Schlingemann et al., Br. J. Ophthalmol., 81:501-512, 1997). In contrast, when VEGF basal expression drops, endothelial cells undergo apoptosis (Alon et al., Nat. Med., 1:1024-1028, 1995) and retinal vasculature regresses, resulting in a reduced retinal blood supply.

Recently, Arden proposed the hypothesis that the high oxygen consumption of dark-adapted rod cells is the driving force of inner retinal hypoxia, with subsequent VEGF production leading to retinal neovascularization in ischemic retinopathies (Arden, G. B., Br. J. Ophthalmol., 85:366-370, 2001). This hypothesis was indirectly supported by the observation that diabetic retinopathy rarely occurs in retinitis pigmentosa patients (Hayakawa et al., Am. J. Ophthalmol., 115:168-173, 1993; Uliss et al., Ophthalmology, 93:1599-1603, 1986; Butner, R. W., Ann. Ophthalmol., 16:861,863-865, 1984; Arden, G. B., Br. J. Ophthalmol., 85:366-370, 2001; Pruett, R. C., Trans. Am. Ophthalmol. Soc., 81:693-735, 1983) and by the clinical success of panretinal photocoagulation, a treatment that destroys a large number of rod photoreceptor cells and reduces intra-ocular VEGF levels (Aiello et al., N. Engl. J. Med., 331:1480-1487, 1994). The present findings show for the first time that degeneration of rod cells leads to a total lack of reactive retinal neovascularization, accompanied by a failure in the expected VEGF up-regulation. Taken together, these observations of Pdeb^{rd1}/Pdeb^{rd1} mice and a human patient afflicted with both diabetes mellitus and retinitis pigmentosa provide direct experimental and mechanistic evidence in support of Arden's hypothesis (Arden, G. B., Br. J. Ophthalmol., 85:366-370, 2001). The data provided here indicate that VEGF is a primary link between rod cell numbers and retinal neovascularization. The data presented show that reducing the metabolic rate of rod cells at critical time windows will improve the incidence of retinopathy of prematurity and slow the progression of diabetic retinopathy in adults. Increased exposure of premature neonates to light will reduce O₂ consumption by rod photoreceptor cells and retinal hypoxia, ultimately improving their retinopathy. These unexpected findings are counter to the current recommendation to decrease ambient light exposure in that setting, which has actually failed to prevent retinopathy of prematurity (Reynolds et al., N. Engi. J. Med., 338:1572-1576 1998).

These observations notwithstanding, VEGF is not the only angiogenic mediator whose production is affected by changes in O₂ tension (Ogata et al., Curr Eyye Res., 16:9-18, 1997; Smith et al., Science, 276:1706-1709, 1997; Khaliq et al., Lab. Invest., 78:109-116, 1998; Yoshida et al., Invest. Ophthalmol. Vis. Sci, 39:1097-1106, 1998; Dawson et al., Science, 285:245-248, 1999; Carmeliet et al., Nat. Med., 7:575-583, 2001). Moreover, VEGF inhibitors and blockers can only partially halt angiogenesis in the retinopathy of prematurity model (Aiello et al., Proc. Natl. Acad. Sci. USA, 92:10457-10561, 1995) and not all of patients with diabetic retinopathy show a rise in VEGF (Aiello et al., N. Engl. J. Med., 331:1480-1487, 1994). Thus, the total absence of retinal neovascularization in homozygous Pdeb^{dr1} mice suggests that the degeneration of photoreceptor cells may have further effects on angiogenesis that are not VEGF-mediated. The data do not rule out a possible role for other angiogenic factors known to be regulated by hypoxia (Ogata et al., Curr Eyye Res., 16:9-18, 1997; Smith et al., Science, 276:1706-1709, 1997; Khaliq et al., Lab. Invest., 78:109-116, 1998; Yoshida et al., Invest. Ophthalmol. Vis. Sci., 39:1097-1106, 1998), such as transforming growth factor-beta, insulin-like growth factor-1, placental growth factor and interleukin-8.

The results disclosed herein highlight the recent awareness that growth factors and inhibitors may be involved in coordinating neural and vascular components of the retina by functioning simultaneously as photoreceptor cell survival factors and endothelial cell regulators. Basic fibroblast growth factor (bFGF) is elevated in Pdeb^{rd1}/Pdeb^{rd1} mice several days before photoreceptor cell death (Gao et al., Dev. Biol., 169:168-184, 1995) and intra-vitreous injection of bFGF delays the onset of photoreceptor cell degeneration in selected animal models (Faktorovich et al., Nature, 347:83-86, 1990). Pigment epithelium-derived factor (PEDF), which is encoded by a gene closely linked to the Pdeb locus (Tombran-Tink et al., Genomics, 19:266-272, 1994), is a survival factor for photoreceptor cells (Cayouette et al., NeurobioL Dis., 6:523-532, 1999) and has been proposed to also play an anti-angiogenic role in the retina (Dawson et al., Science, 285:245-248, 1999). Given that PEDF concentration is highest in the matrix surrounding the photoreceptor cell layer (Dawson et al., Science, 285:245-248, 1999; Becerra, B. P, Chemistry and Biology of Serpins Kluwer Academic Publishers, Boston, 1997) that undergoes apoptosis in Pdeb^{rd1}/Pdeb^{rd1} mice Chang et al., Neuron, 11:595-605, 1993; Portera-Cailliau et al., Proc. Nail. Acad. Sci. USA 91:974-978, 1994), one might expect that a loss of PEDF would be correlated with an increase rather than a decrease in retinal angiogenesis.

In our system, immunostaining failed to show a correlation between PEDF and neovascularization (data not shown) and suggest that PEDF does not play a major role in the phenomenon described here. However, it is possible that PEDF or other angiogenesis inhibitors are released during the photoreceptor cell apoptotic process, which may contribute to the lack of retinal neovascularization. Roles for additional factors are suggested by the dramatic but unexplained variations in timing of retinal capillary growth into the pigment epithelial cell layer among mutant mice that share a similarity in timing of photoreceptor cell degeneration (Nishikawa et al., Exp. Eye Res., 67:509-515, 1998). Finally, the neurotransmitter dopamine has been recently shown to inhibit VEGF-induced angiogenesis (Basu et al., Nat. Med., 7:569-574, 2001). However, dopamine synthesis and utilization are known to be suppressed at least in some mouse models of retinal degeneration (Nir et al., Brain Res., 884:13-22, 2000).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of reducing and/or preventing retinopathy of prematurity comprising: increasing exposure of a premature neonate to light.

2. The method of claim 1, further comprising administering anti-angiogenic compounds to the neonate.

3. The method of claim 2, wherein the anti-angiogenic compounds are administered directly to the eyes of the neonate.

4. The method of claim 2, wherein the anti-angiogenic compound is bound to a ligand which selectively binds angiogenic cells of the retinal vasculature.

5. The method of claim 1, further comprising administering VEGF inhibitors to the neonate.

6. The method of claim 1, further comprising administering inhibitors of rod cell oxidative metabolism to the neonate.

7. The method of claim 6, wherein the inhibitor of rod cell oxidative metabolism is selected from the group consisting of rotenone, amytal, antimycin A, oligomycin, H-8, Rp-8-bromo-cGMP, Rp-8-pCPT-cGMPS, Rp-8-Br-cGMPS, a barbiturate, an anesthetic, lidocaine and procaine.

8. The method of claim 1, wherein exposure of the premature neonate to light reduces oxygen consumption of the neonate's rod photoreceptor cells.

9. A method of reducing and/or preventing diabetic retinopathy comprising: increasing exposure of a diabetic individual to light.

10. The method of claim 9, further comprising administering anti-angiogenic compounds to the individual.

11. The method of claim 10, wherein the anti-angiogenic compounds are administered directly to the eyes of the individual.

12. The method of claim 9, further comprising administering VEGF inhibitors to the individual.

13. The method of claim 9, further comprising administering inhibitors of rod cell oxidative metabolism to the individual.

14. A method of identifying inhibitors of retinal neovascularization comprising:
a) exposing C57BL/6+/+wt (wild-type) mice and/or Pdeb^{rd1}/Pdeb^{rd1} mutant mice to hyperoxia;
b) treating the mice with a putative inhibitor of retinal neovascularization;
c) assaying for inhibition of retinal neovascularization.

15. The method of claim 14, wherein the assay comprises histologic analysis of blood vessel protrusion into the vitreous; determining oxidative metabolism of rod cells; and/or measuring VEGF expression in rod cells.

16. The method of claim 14, further comprising isolating rod cells from the mice and treating the isolated rod cells with the putative inhibitor.

17. The method of claim 16, further comprising performing the assay on the isolated rod cells.

18. A method of preventing retinal neovascularization comprising: administering a compound which decreases the oxidative metabolism of rod cells and preventing retinal neovascularization.

19. The method of claim 18, further comprising exposing the retina to increased light levels.

20. A pharmaceutical composition comprising an inhibitor of rod cell oxidative metabolism.
